(19) United States
(12) Patent Application Publication
(10) Pub. No.: US 2006/0228736 A1
(43) Pub. Date: Oct. 12, 2006

(54) Blood testing method, nucleic acid extraction method, and nucleic acid extraction device

(75) Inventors: Toshinari Sakurai, Hitachinaka (JP); Yoshihiro Yamashita, Hitachinaka (JP)

Correspondence Address: MCDERMOTT WILL & EMERY LLP, 600 13TH STREET, N.W., WASHINGTON, DC, 20005-3096, US

(73) Assignee: HITACHI HIGH- TECHNOLOGIES CORPORATION

(21) Appl. No.: 11/397,732

(22) Filed: Apr. 5, 2006

(30) Foreign Application Priority Data
Apr. 6, 2005 (JP) 2005-110037

(51) Int. Cl. C12Q 1/68; G06F 19/00

(52) U.S. Cl. 435/6; 702/20

(57) ABSTRACT

The present invention provides a method which makes it possible to extract both a liquid sample for a biochemical examination and an immunological test and a blood cell sample for a nucleic acid test from one blood sample. Isolation of blood cells is carried out for the blood obtained by blood collection. To the isolated blood cell component, a solution is added, and furthermore, which is suspended. Thus, the obtained blood cell sample is freeze-preserved, or used for extraction of nucleic acid. A nucleic acid test is carried out with the use of the extracted nucleic acid. On the other hand, the isolated plasma component is freeze-preserved, or provided for a biochemical examination and an immunological test.

### BACKGROUND OF THE INVENTION

1. Field of the Invention

The present invention relates to a method for assay of withdrawn blood, a nucleic acid extraction method, and a nucleic acid extraction device.

2. Background Art

In recent years, the development of a clinical chemistry automated analyzer and an immunological automated analyzer led to easily performing an assay with serum or plasma isolated from the blood as a sample. The results from these assays are used directly or indirectly as useful information for medical care or treatment of diseases.

On the other hand, various nucleic acid analysis techniques have been developed with the progress of molecular biology. Many pathogenic genes have been isolated or identified by these nucleic acid analysis techniques. Accomplishments in the nucleic acid analysis have been used in medical care or treatment of diseases. In addition, molecular biological techniques have been introduced to the assay performed in the field of healthcare, which makes possible an assay which was impossible in the past.

The progress in nucleic acid analysis techniques depends greatly on a nucleic acid amplification method, especially, the PCR (polymerase chain reaction, Saiki et al., Science, 239, 487-491 (1988)). With the PCR method, it is possible to sequence-specifically amplify nucleic acid in a sample. Accordingly, for example, by amplifying and detecting nucleic acid which is a viral gene, it is possible to prove indirectly the presence of virus which is present in trace amount in the serum.

The PCR method uses the blood as a sample, which is easy to collect. However, the PCR method poses some problems when it is used for routine clinical examination. Especially among them, the processes of extraction and purification for nucleic acid in the pretreatment have been pointed out to be important in the maintenance of high-purity nucleic acids (Ohshima et al., JJCLA, 22(2), 145-150(1997)). This is because the inhibitor, which has not been possible to remove in the purification process of nucleic acid, has an influence on the purity of nucleic acid. In the case of using the blood as a sample, hemoglobin in the blood is known as such an inhibitory agent. Therefore, it is needed to select suitable extraction and purification processes of nucleic acid.

### SUMMARY OF THE INVENTION

In the past, when performing a biochemical examination, an immunological test and a nucleic acid test with the use of the blood of a patient, the blood needed to be collected separately for the biochemical examination and the immunological test, and the nucleic acid test. The reason is because it was difficult to extract a blood cell sample for nucleic acid extraction in the nucleic acid test from the residue of the serum and the plasma, which had been adjusted for the biochemical examination and the immunological test.

As a preparation method of the blood sample for nucleic acid extraction, a method is known wherein red blood cell is dissolved by an agent in advance, and the buffy coat is prepared, which contains white blood cell in a large amount. However, with this method, the liquid component can not be used as a sample for a biochemical examination or an immunological test although a sample for nucleic acid extraction can be obtained due to the dissolution of red blood cell.

An object of the present invention is to provide a method which makes it possible to extract both a liquid sample for a biochemical examination and an immunological test and a blood cell sample for a nucleic acid test from one blood sample.

Furthermore, another object of the present invention is to provide a method of making comprehensive judgment for the health of a patient by carrying out a biochemical examination, an immunological test and a nucleic acid test from one blood sample.

According to the present invention, the isolation of blood cells is carried out for the blood obtained by blood collection. To the isolated blood cell component, a solution is added, and furthermore, which is suspended. Thus obtained blood cell sample is freeze-preserved, or used for extraction of nucleic acid. A nucleic acid test is carried out with the use of the extracted nucleic acid. On the other hand, the isolated plasma component is freeze-preserved, or provided for a biochemical examination and an immunological test.

According to the present invention, it possible to extract both a serum or plasma sample for a biochemical examination and an immunological test and a blood cell sample for a nucleic acid test from one blood sample.

### BRIEF DESCRIPTION OF THE DRAWINGS

FIG. 1 is a view showing a method of extracting the plasma and nucleic acid from the blood according to the present invention.

FIG. 2 is a view showing a method of extracting the plasma and nucleic acid from the blood according to the present invention, carrying out an assay and making a comprehensive judgment.

FIG. 3 is a view showing a method of extracting the plasma from the blood according to the present invention, carrying out a primary assay, and then extracting nucleic acid and carrying out a nucleic acid test according to the results of the assay, and making a comprehensive judgment.

FIG. 4 is a view showing the external appearance of an apparatus for extracting nucleic acid which is used in an experiment for nucleic acid extraction.

FIG. 5 is an exploded view showing an apparatus for extracting nucleic acid which is used in an experiment for nucleic acid extraction.

FIG. 6 is a view showing the structure of the solid phase carrier unit which is used in an experiment for nucleic acid extraction.

FIG. 7 is a viewing showing the results of the experiment for nucleic acid extraction.

### DESCRIPTION OF THE PREFERRED EMBODIMENTS

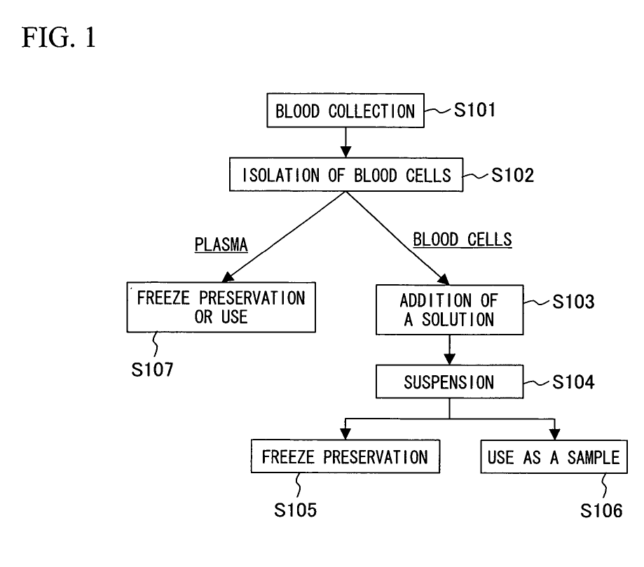

Procedures of the extraction method for the blood components according to the present invention will be explained with reference to FIG. 1. First, blood collection is carried out in Step S101. A blood collection method includes a method through a vacuum blood collection tube, a method through a blood collection syringe and the like. An anticoagulant may be added to the whole blood obtained by blood collection. The anticoagulant includes EDTA, citric acid, heparin, sodium fluoride and the like. Isolation of blood cells is carried out in Step S102. Specifically, blood cells are isolated from the plasma. The isolation of blood cells includes a method through centrifugation, a method through a membrane for isolation of blood cells and the like.

For example, in the case of the whole blood sample immediately after blood collection at 15,000 to 2,000 g, it is possible to isolate blood cells from the plasma by 10 to 15 minute centrifugation.

To the isolated blood cell component, a solution is added in Step S1103. The solution to be added may be a solution which does not aggregate the blood cell component remarkably, and it has preferably pH of 6 or more, and further has the salt concentration of 110 mmol/L or more. Such solution to be added includes a buffer solution which has pH buffering action such as a phosphoric acid solution, Tris solution or the like, and a salt solution of the concentration where no denaturation is given to the blood cell component, such as normal saline solution. The blood cell component to which the solution is added, is suspended in Step S104. A method for the suspension includes a method by a test tube mixer, a method by a shaking stirrer and the like.

Freeze preservation is performed in Step S105 in the case of freeze preservation. The freeze preservation is desirably performed at the temperature of −80° C. or less. Thus, this example is characterized in that the blood cell component after isolation is frozen, not the blood immediately after blood collection. In the case of no freeze preservation, it is used as a sample with advancing to Step S106. For example, the extraction for nucleic acid is carried out with the use of the blood cell component.

The isolated plasma is preserved or used as a sample in Step S107. In the case of preservation, it is desirably freeze-preserved at the temperature of −20° C. or less, but when it is provided for a nucleic acid test such as HCV RNA detection, it is desirably preserved at the temperature of −80° C. or less.

Thus, in this example, it is possible to prepare both a sample of the blood cell component and a sample of the plasma component at the same time from one whole blood sample.

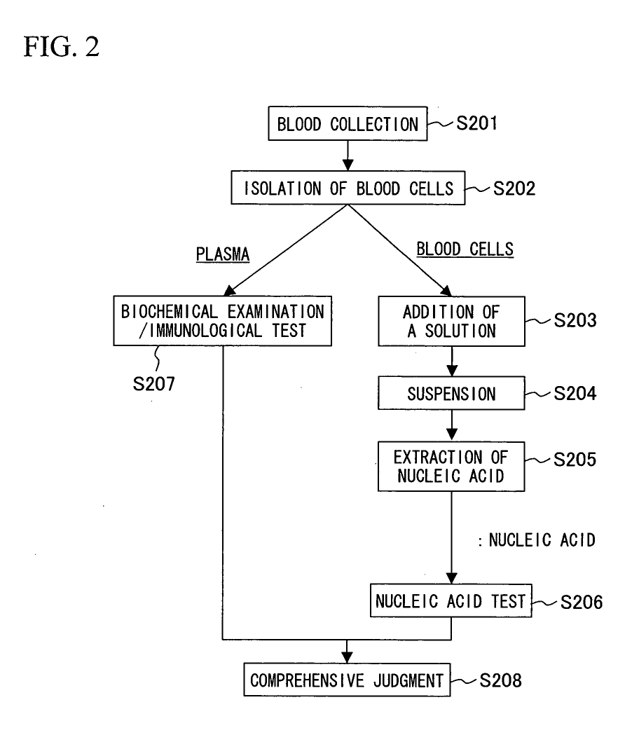

Procedures of carrying out a biochemical examination, an immunological test, and a nucleic acid test with the use of the extraction method for the blood components according to the invention, and carrying out comprehensive judgment for the health of the blood-collected person will be explained with reference to FIG. 2. Herein, a case will be explained where no freeze preservation is carried out. Steps S201 to S204 may be similar to Steps S101 to S104 in FIG. 1. Specifically, blood collection is carried out in Step S201. Isolation of blood cells is carried out in Step S202. A solution is added to the isolated blood cell component in Step S203. The blood cell component to which the solution is added is suspended in Step S204.

Nucleic acid extraction is carried out in Step S205. Extraction of nucleic acid may be performed with the use of an existing technique below. For example, with the nucleic acid extraction method described in JP-A No. 2-289596, first, a sample which contains nucleic acid is mixed with a chaotropic substance and the nucleic acid binding-solid phase so that the nucleic acid is made to bind to the nucleic acid binding-solid phase. Then, the nucleic acid binding-solid phase to which nucleic acid is bound, is isolated from the liquid, washed with a buffer solution for washing, and then washed with alcohol and acetone. With the nucleic acid extraction method described in JP-A No. 6-205676, the whole blood is treated with a surfactant agent and a protease, and brought in contact with a chaotropic agent to isolate DNA. Then, a DNA strand is precipitated by adding alcohols. With the nucleic acid extraction method described in JP-A No. 11-266864, a mixture which contains nucleic acid and a binding promoter is aspirated into a chip for capture of nucleic acid, and nucleic acid is captured by nucleic acid binding-solid phase which is installed in the chip for capture of nucleic acid.

A nucleic acid test is carried out with the use of the extracted nucleic acid in Step S206. On the other hand, a biochemical examination and an immunological test are carried out for the isolated plasma as a sample in Step S207. Comprehensive judgment is carried out in Step S208. Comprehensive judgment is carried out from the assay results of the biochemical examination, the immunological test and the nucleic acid test.

The blood collection in Step S201 and isolation of blood cells in S202 are performed in blood collection facilities such as a hospital and the like. The isolated blood cell component is delivered to a nucleic acid test center where a nucleic acid test is carried out. The nucleic acid test center carries out the treatments in Steps S203 to S206. Specifically, the extraction for nucleic acid is carried out with the use of the blood cell component, and a nucleic acid test is carried out with the use of the extracted nucleic acid. The isolated plasma component is delivered to an assay center where a biochemical examination and an immunological test are carried out. This assay center carries out the biochemical examination and the immunological test in Step S207.

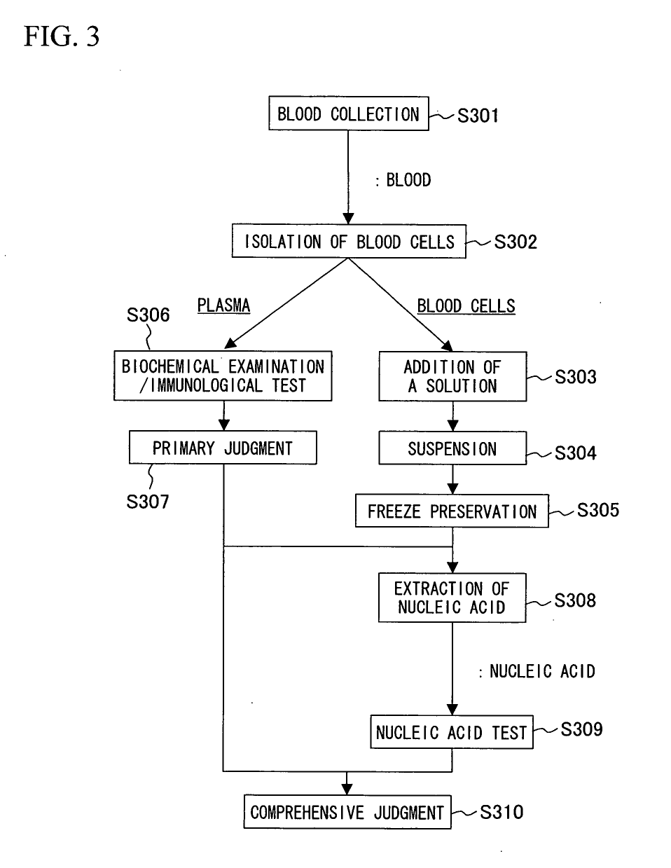

Procedures of carrying out a biochemical examination, immunological test and nucleic acid test with the use of the extraction method for the blood component according to the present invention, and carrying out comprehensive judgment for the health of the blood-collected person will be explained with reference to FIG. 3. Herein, a case will be explained where freeze preservation is carried out. Steps S301 to S305 may be similar to Steps S101 to S105 in FIG. 1. Specifically, blood collection is carried out in Step S301. Isolation of blood cells is carried out in Step S302. A solution is added to the isolated blood cell component in Step S303. The blood cell component, to which the solution has been added, is suspended in Step S304. The blood cell component is frozen in Step S305.

A biochemical examination and an immunological test are carried out for the isolated plasma as a sample in Step S306. Primary judgment is carried out for the health of the blood-collected person from the assay results of the biochemical examination and the immunological test in Step S307. It is advanced to Step S308 when it has been determined that a nucleic acid test needs to be carried out from the results of the primary judgment. Nucleic acid extraction is carried out in Step S308. The frozen blood cell component is thawed, and existing nucleic acid extraction technique may be used. A nucleic acid test is carried out with the use of the extracted nucleic acid in Step S309. If the nucleic acid test is completed, it is advanced to Step S310.

It is advanced to Step S310 when it has been determined that no nucleic acid test needs to be carried out from the results of the primary judgment. Comprehensive judgment is carried out in Step S310. The comprehensive judgment is carried out from the assay results of the biochemical examination and the immunological test in case that no nucleic acid test is carried out. Comprehensive judgment is carried out from the assay results of the biochemical examination, immunological test and nucleic acid test in case that the nucleic acid test has been carried out. In this example, a nucleic acid test, which is relatively expensive, is performed only if necessary, which allows reduction of the price for the comprehensive judgment.

The blood collection in Step S301 and isolation of blood cells in S302 are performed in blood collection facilities such as a hospital. The isolated blood cell component is delivered, for example, to a nucleic acid test center where a nucleic acid test is carried out. The nucleic acid test center carries out the treatments of Steps S303 to S305 and S308 to S309. Specifically, the blood cell component is frozen, nucleic acid extraction is carried out if necessary, and a nucleic acid test is carried out with the use of the extracted nucleic acid. The isolated plasma component is delivered, for example, to an assay center where a biochemical examination and an immunological test are carried out. This assay center carries out the biochemical examination and the immunological test in Step S306. The primary judgment in Step S307 and comprehensive judgment in Step S310 are performed, for example, by a doctor at a hospital.

### EXAMPLES

Examples of the present invention will be explained herein below. In this Example, extraction of the plasma and blood cells were carried out according to the flowchart in FIG. 1, and extraction for nucleic acid was carried out from the blood cell component. A vacuum blood collection tube (Venoject II, manufactured by Terumo Corporation) which encompasses EDTA as an anticoagulant was used in the blood collection of Step S101, and the blood was mixed with the anticoagulant by tumble mixing. In the isolation of the blood cells in Step S102, a centrifuge (CR5B2, manufactured by Hitachi Koki Co., Ltd.) was used, and a vacuum blood collection tube was centrifugally rotated in 3,000 rpm for 10 minutes. The supernatant was preparatively isolated by a micropipette (Reference 1000, manufactured by Eppendorf Co., Ltd.) wherein a disposable tube of 1 mL volume is mounted, to give the plasma. The plasma preparatively isolated was put into a polypropylene centrifuging tube (manufactured by Falcon Co., Ltd.) of 15 mL volume and preserved at −20° C.

The plasma preparatively isolated and the same amount of a solution were added to the blood cell component of the residue in the addition of a solution in Step S1103. It was suspended by a test tube mixer (Vortex GENIE2, Scientific Industries) in the suspension of Step S104, which was taken as a sample for nucleic acid extraction.

Then, the extraction for nucleic acid will be explained. The method described in JP-A No. 11-266864 was used for extraction of nucleic acid. Herein, a syringe for extraction will be explained, which is manufactured for extraction of nucleic acid by the present inventors.

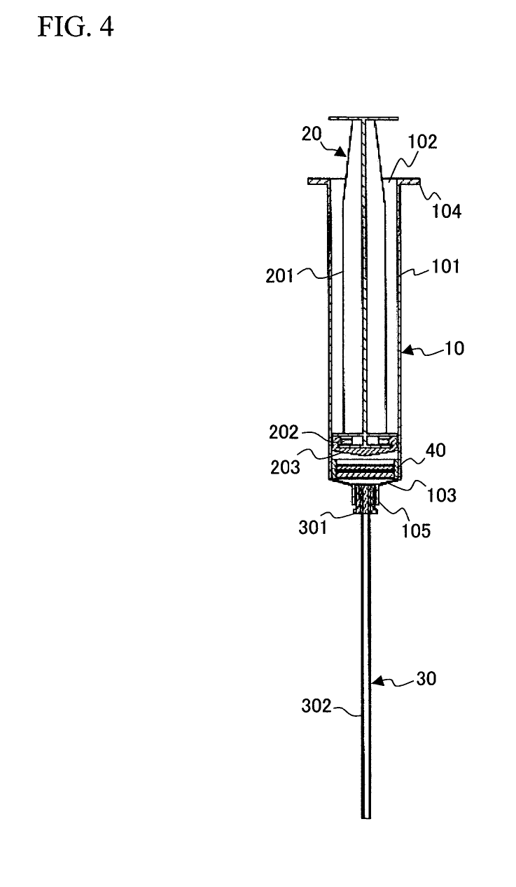

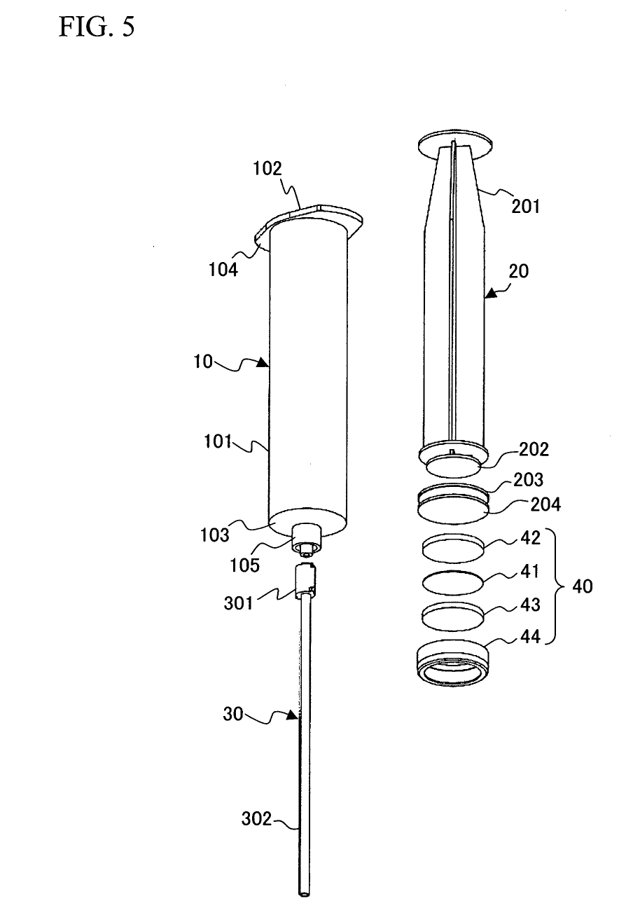

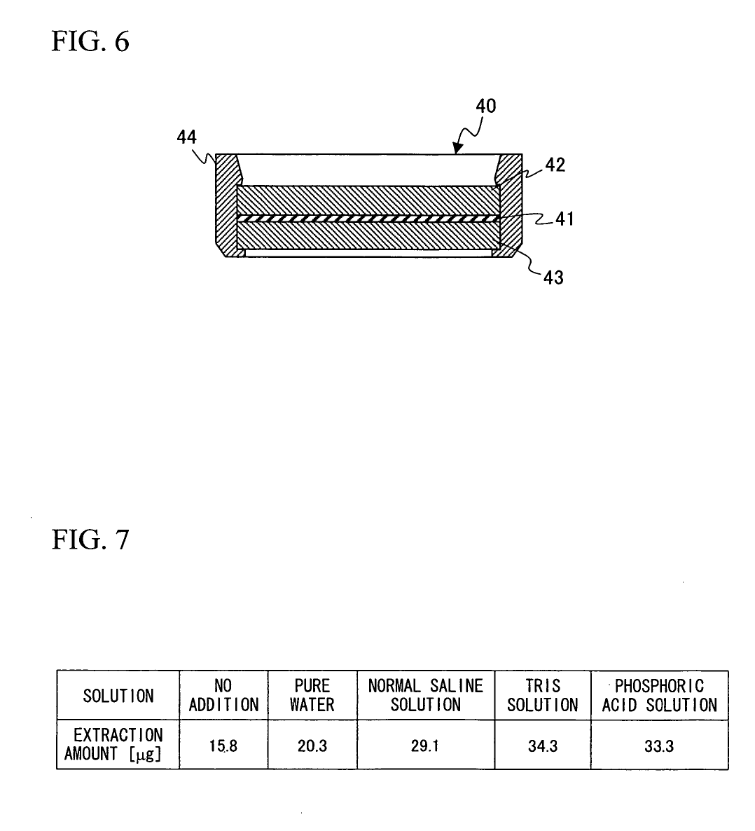

FIG. 4 shows the external appearance of the syringe for extraction, and FIG. 5 shows the structure of the syringe for extraction. FIG. 6 shows the structure of the solid phase carrier unit. The syringe for extraction in this example comprises a syringe main body 10, a plunger 20, a nozzle 30 and a solid phase carrier unit 40. The nozzle side is called as the lower side, and the plunger side as the upper side.

The syringe main body 10 has a cylindrical part 101 of a cylindrical form, an opening part 102 on the upper end, a bottom part 103 on the lower end, a holding part 104 of a flange form installed around the opening part 102 and a connection part 105 for connecting to the nozzle which is installed in the bottom part 103. A 30 mL syringe (the lock type) manufactured by Terumo Corporation was used as the syringe main body 10.

As shown in FIG. 5, the plunger 20 has a plunger main body 201 and a seal piece 203. The seal piece 203 is formed as a member separate from the plunger main body 201, and mounted on a mounting part 202 on the lower end of the plunger main body 201. The seal piece 203 has a conical projection 204 on the lower end.

The nozzle 30 has a connection part 301 on the upper end and a tubular part 302 which extends downward therefrom. The connection part 301 of the nozzle and the connection part 105 of the syringe main body are connected by a screw.

The syringe main body 10 and the plunger main body 201 are formed from polypropylene, the seal piece 203 from rubber, and the nozzle 30 from the PEEK material.

The solid phase carrier unit 40 is constituted by a disc-like solid phase carrier 41, two disc-like holding members 42 and 43 arranged on the upper side and the lower side of the solid phase carrier 41, and a cylindrical holder 44 as shown in FIG. 5 and FIG. 6. The holding members 42 and 43 and the cylindrical holder 44 are formed from polypropylene.

The solid phase carrier 41 was manufactured by punching a glass fiber filter manufactured by Whatman into a disc form with a cutter of a punch form.

Then, a reagent used for extraction of nucleic acid will be explained. Herein, 6 kinds of reagents below were used.

The first reagent: a solution of a protease

The second reagent: a buffer solution which contains guanidine hydrochloride, a surfactant agent and MES

The third reagent: an ether solution

The fourth reagent: a buffer solution which contains guanidine hydrochloride, a surfactant agent and MES

The fifth reagent: a buffer solution which contains an acetate salt and ethanol

The sixth reagent: a Tris buffer solution

First, the suspended blood cell component, which is a sample for nucleic acid extraction, was weighed in 6 mL, and preparatively isolated into a 50 mL centrifuging tube. To this 50 mL centrifuging tube, 0.6 mL of the first reagent was added, and the mixture was stirred by a test tube mixer. Then, 7.2 mL of the second reagent was added, and the mixture was stirred again by the test tube mixer. This 50 mL centrifuging tube was warmed as capped for 20 minutes with a block incubator at 80° C. (DTU-1C, manufactured by Tietech Co., Ltd.), and then taken out from the block incubator and left for the prescribed time at room temperature.

Then, the cap of the 50 mL centrifuging tube was opened, 6.2 mL of the third reagent was added thereto, and it was stirred with a test tube mixer to give a mixture. Nucleic acid extraction was carried out from this mixture with the use of the syringe apparatus for extraction of FIG. 4.

First, the plunger 20 was moved upward with the tip part of the nozzle 30 soaked in the mixture in the 50 mL centrifuging tube, and the mixture was aspirated into the inside of the extraction syringe. Thereby, the mixture was passed through the solid phase carrier 41. Then, the plunger 20 was moved downward, and the mixture was discharged into the outside of the extraction syringe. Thereby, the mixture was passed again through the solid phase carrier 41. Such treatment was performed 10 times of passing the mixture through the solid phase carrier 41 by the procedures of the aspiration and discharge. Finally, the mixture was disposed into a 50 mL centrifuging tube in the total amount.

Then, by similar procedures of aspiration and discharge for the plunger 20, a treatment of passing 25 mL of the fourth reagent through the solid phase carrier 41 was carried out three times, and furthermore, a treatment of passing the newly changed fourth reagent through the solid phase carrier 41 was carried out three times. For the fifth reagent, a treatment of passing it through the solid phase carrier 41 was also carried out by procedures of aspiration and discharge, similarly to the case of the fourth reagent. Then, for 1.2 mL of the sixth reagent, a treatment of passing it through the solid phase carrier 41 was also carried out ten times by similar procedures of aspiration and discharge, to extract the whole amount.

FIG. 7 shows the amount of nucleic acid extracted from 1 mL of the blood cell component of the same person, by the nucleic acid extraction method described above with changing the kind of the solution to be added to the blood cell component.

From the results of FIG. 7, it was understood that nucleic acid extraction rate increases by adding a solution to the blood cell component and suspending it in the solution. It was especially understood that a buffer solution is very suitable as the solution to be added. In addition, a solution of nucleic acid extracted herein was subjected to 0.4% agarose gel electrophoresis, and as a result, a band was found which represents human genome DNA. Furthermore, the PCR of a beta-globin region was carried out with a solution of the extracted nucleic acid as a template, and as a result, normal gene amplification was found.

From the above, according to the present method, it was possible to extract the plasma component and nucleic acid from the same blood collection tube, and furthermore, it was possible to carry out the assay suitably by the extracted component.

The examples of the present invention have been explained above, but the present invention is not limited to these examples, and it is easily understood by those skilled in the art that various modifications for them can be made within the scope of the invention described in Claims.

What is claimed is:

1. A blood testing method comprising:
isolating a collection of blood sample into a liquid component and a blood cell component to produce a liquid sample which comprises the liquid component and a blood cell sample which comprises the blood cell component from one collection of blood sample;
adding a predetermined solution to the blood cell sample to produce a sample for nucleic acid extraction;
carrying out at least one of a biochemical examination and an immunological test with the use of the liquid sample; and
producing data for judging the health of the blood-collected person of the collection of blood sample from the results of at least one of the biochemical examination and the immunological test performed above.

2. The blood testing method according to claim 1, further comprising:
extracting nucleic acid with the use of the sample for nucleic acid extraction; and
carrying out a nucleic acid test with the use of the extracted nucleic acid,
wherein the data for judging the health is produced on the basis of the results of at least one of the biochemical examination and the immunological test performed above, and the results of the nucleic acid test.

3. The blood testing method according to claim 1, wherein the predetermined solution has pH of 6 or more and further has the salt concentration of 10 mmol/L or more.

4. The blood testing method according to claim 1, wherein the predetermined solution is a buffer solution which has pH buffering action.

5. The blood testing method according to claim 1, wherein the predetermined solution is a phosphoric acid solution, Tris solution, or normal saline solution.

6. The blood testing method according to claim 1, further comprising:
freeze-preserving the sample for nucleic acid extraction.

7. The blood testing method according to claim 1, further comprising:
freeze-preserving the liquid sample.

8. The blood testing method according to claim 1, wherein the sample for nucleic acid extraction is produced by adding a predetermined solution to the blood cell sample, and further suspending it in the solution.

9. A nucleic acid extraction method comprising:
removing the liquid component from the blood to thereby produce a sample for nucleic acid extraction which comprises only a blood cell component;
adding a predetermined solution to the sample for nucleic acid extraction; and
extracting nucleic acid with the use of the sample for nucleic acid extraction to which the solution has been added.

10. The nucleic acid extraction method according to claim 9, wherein the predetermined solution has pH of 6 or more and further has the salt concentration of 10 mmol/L or more.

11. The nucleic acid extraction method according to claim 9, wherein the predetermined solution is a buffer solution which has pH buffering action.

12. The nucleic acid extraction method according to claim 9, wherein the predetermined solution is a phosphoric acid solution, Tris solution, or normal saline solution.

13. The nucleic acid extraction method according to claim 9, wherein the sample for nucleic acid extraction is produced by adding a predetermined solution to the blood cell sample, and further suspending it in the solution.

14. The nucleic acid extraction method according to claim 9, further comprising:
freeze-preserving the sample for nucleic acid extraction.

15. A nucleic acid extraction device comprising:
a solution to be added to a sample which comprises only a blood cell component produced by removing a liquid component from the blood;
a reagent to be used for extracting nucleic acid from the sample for nucleic acid extraction; and
a nucleic acid extraction syringe for extracting nucleic acid from the sample for nucleic acid extraction.

16. The nucleic acid extraction device according to claim 15, wherein the predetermined solution has pH of 6 or more and further has the salt concentration of 10 mmol/L or more.

17. The nucleic acid extraction device according to claim 15, wherein the predetermined solution is a buffer solution which has pH buffering action.

18. The nucleic acid extraction device according to claim 15, wherein the predetermined solution is a phosphoric acid solution, Tris solution, or normal saline solution.

19. The nucleic acid extraction device according to claim 15, wherein the nucleic acid extraction syringe has a syringe main body, a plunger, a nozzle and a solid phase carrier unit, and the solid phase carrier unit is held in the inside of the syringe main body, and has a circular glass fiber filter and holding members which sandwich the filter paper from both sides.

20. The nucleic acid extraction device according to claim 15, wherein the reagent comprises at least one of the first reagent which is a solution of a protease, the second reagent which is a buffer solution which comprises guanidine hydrochloride, a surfactant agent and MES, the third reagent which is an ether solution, the fourth reagent which is a buffer solution which comprises guanidine hydrochloride, a surfactant agent and MES, the fifth reagent which is a buffer solution which comprises an acetate salt and ethanol, and the sixth reagent which is a Tris buffer solution.
